(19) United States
(12) Patent Application Publication
(10) Pub. No.: US 2024/0333000 A1
(43) Pub. Date: Oct. 3, 2024

(54) Smart ring with electrodes, and glue filling jig and packaging process thereof

(71) Applicant: Guangdong Jiu Zhi Technology Co., Ltd., Zhongshan City (CN)

(72) Inventors: Min Wang, Shanghai City (CN); Qianjin Feng, Zhongshan City (CN); Minghua Shi, Huayuan County (CN)

(73) Assignee: Guangdong Jiu Zhi Technology Co., Ltd., Zhongshan City (CN)

(21) Appl. No.: 18/619,111

(22) Filed: Mar. 27, 2024

(30) Foreign Application Priority Data
Mar. 29, 2023 (CN) 202310321801.7

(51) Int. Cl. H02J 7/00; A61B 5/00; A61B 5/256; H05K 1/02; H05K 7/14

(52) U.S. Cl. CPC H02J 7/0042; A61B 5/256; A61B 5/6826; H05K 1/0277; H05K 7/1427; A61B 2560/0214; A61B 2560/0468; A61B 2562/125; A61B 2562/166

(57) ABSTRACT

The present invention discloses a smart ring with electrodes, a glue filling jig, and its packaging process thereof. It includes a ring outer shell which is a ring with an inner opening, a ring inner shell mounted in the opening, and a cavity defined by the inner and outer shells. A flexible printed circuit board is installed inside the cavity. The present invention integrates all components into a complete structure by filling the cavity with high-permeability biocompatible glue, significantly improving waterproofing. A specially made jig controllably blocks part of the glue path, solving the issue that electronic components must be lower than the cavity height in the glue filling process. This makes the invention more suitable for manufacturing smart rings with electrodes.

### REFERENCE TO PRIOR APPLICATION

This application claims priority to Chinese Patent Application 202310321801.7, filed on Mar. 29, 2023, which is incorporated herein by reference.

### TECHNICAL FIELD

The present invention relates to the technical field of smart rings, in particular to a smart ring with electrodes, and a glue filling jig and a packaging process thereof.

### BACKGROUND

Smart rings designed for health monitoring have become a rising star in the wearable electronic device market because of the convenient, feelingless and continuous measurement and other characteristics. To meet user needs in various scenarios, the packaging process for smart rings must achieve both high waterproofness and excellent comfort for wearers.

There are the following two common packaging processes for smart rings:

A: The first method is molded inner and outer shell assembly. In this approach, electronic components are placed inside the casing cavity. An O-ring is installed for sealing, and then the inner shell is mounted into a pre-existing slot. The assembly is secured using screws or fasteners, completing the encapsulation.

B: The second is the glue filling process, as used in U.S. Pat. No. 10,893,833 B2, that is, the electronic components are first arranged in the molded outer shell cavity, and the thickness of electronic components must be smaller than the thickness of the outer shell cavity, and then a plastic filling material is used to fill the ring cavity till the electronic components are completely covered and sealed.

A: Assembly of molded inner and outer shells. Rings produced through the process have good rigidity, however, the complex structural parts can increase the processing cost on the one hand, and the method of fixing through screws or buckle structures cannot meet the high waterproofness; on the other hand, the complex structural parts can also increase the mass and thicknesses of the rings and reduce the wearing comfort, which is contrary to the original design intention of smart rings for comfortable, continuous, and feelingless health monitoring.

B: Traditional glue filling process. Smart rings produced through such process have a simple structure and high waterproof properties, but require that the thickness of the electronic components must be smaller than the thickness of the cavity, which makes it difficult to assemble electronic components including electrodes, such as magnetic charging metal electrodes, or health detection electrodes (such as electrocardiogram or electrodermal detection electrodes) required to be in contact with the human body. The aforementioned electrodes need to be stably connected to an electronic circuit board. At the same time, part of the electrodes are required to be exposed to the outer surface of the ring, which cannot be satisfied by the traditional glue filling process.

### SUMMARY

The purpose of the present invention is to provide a smart ring containing electrodes, and a glue filling jig and a packaging process thereof, to solve the problems raised in the aforementioned background.

In order to achieve the aforementioned purpose, the present invention provides the following technical solution: a smart ring containing electrodes, comprising
a ring outer shell which is a ring with an inner opening;
a ring inner shell which is arranged inside the ring outer shell;
a ring outer shell cavity which is the cavity defined by the ring outer shell and the ring inner shell;
a flexible printed circuit board which is mounted inside the ring outer shell cavity;
electronic elements which are arranged on the flexible printed circuit board and comprise a sensing component, a processing component, a Bluetooth component, and a charging component;
a battery which is arranged inside the ring outer shell cavity, and electrically connected with the flexible printed circuit board to supply power to the flexible printed circuit board;
preferably, a charging component includes a charging management unit, a No. 1 charging metal electrode and a No. 2 charging metal electrode, wherein the No. 1 charging metal electrode and the No. 2 charging metal electrode are symmetrically arranged on the outer surface of the flexible printed circuit board.

Preferably, an insulating element is mounted on the inner wall of the ring outer shell, and positioned closely to the flexible printed circuit board.

Preferably, a mounting area of the insulating element covers the area accessible to the flexible printed circuit board, blocking the electrical connection between the electronic components and the ring outer shell, and the insulating element is made of insulating glue or ultra-thin insulated rubber tape.

Preferably, the ring outer shell is made of one of titanium, steel, copper, gold, silver, alloys or ceramic non-alloys, and the ring inner shell is made of flexible glue which can be composed of oxygen resin, polyethylene, cyclohexene oxide or a type of photosensitive adhesive.

A glue filling jig for a smart ring containing electrodes, comprising
a supporting frame which has a rectangular cavity with a top opening;
a No. 1 elastic die which is arranged inside the supporting frame;
a spacing ring groove which is formed inside the No. 1 elastic die and used for loading the smart ring that has not been sealed with glue;
a glue filling space which is formed between the unsealed smart ring and the inner wall of the spacing ring groove and used for feeding glue and shaping the ring inner shell;
a spacing part which is arranged on the geometric center of the supporting frame.

Preferably, a No. 1 spacing column and a No. 2 spacing column are arranged on the spacing part, wherein a No. 2 elastic die is arranged at one end of the No. 1 spacing column and one end of the No. 2 spacing column.

Preferably, the spacing part and the inner wall of the supporting frame form a spacing clamping groove.

A packaging process of a smart ring containing electrodes, comprising the following steps:
mounting the insulating element in a corresponding area of the inner wall of the ring outer shell;
dispensing glue in a corresponding position of the flexible printed circuit board on the inner wall of the ring outer shell through a dispensing process, and fixing the flexible printed circuit board and the battery in the ring outer shell cavity.
mounting the unsealed smart ring in the glue filling jig according to any one of the claims, and filling glue to the glue filling space of the glue filling jig with a special syringe filled with glue, to ensure that the glue completely fills the inner cavity of the ring outer shell;
leaving the jig standing for hours after glue filling is completed, securing the position of the flexible printed circuit board and exposing part of the electrodes, and performing demolding to form a glue ring inner shell after the glue is completely reacted and solidified.

Compared with the prior art, the present invention has the beneficial effects that the ring outer shell, the internal electronic element and the ring inner shell are integrated into a complete structure through the method of filling the ring outer shell cavity with high permeability biocompatible glue to form the ring inner shell, and the waterproofness of the smart ring is improved. Additionally, part of a glue flowing path is controllably blocked through the jig, addressing the challenge that electronic components are required to be lower than the height of the ring outer shell cavity in the glue filling process. This invention is particularly suitable for the manufacturing and machining of smart rings containing electrodes;
adding the isolating element to the inner wall of the ring inner shell isolates the flexible printed circuit board from the ring outer shell, reduces the risk of short circuit and improves the ring yield; high waterproofness and wearing comfort of the smart ring are achieved, meanwhile good contact performance of electrodes is ensured, the yield is improved, and the production cost is reduced.

### BRIEF DESCRIPTION OF DRAWINGS

FIG. 1 is a schematic structural diagram of the smart ring of the present invention;

FIG. 2 is a top view of the glue filling jig of the present invention;

FIG. 3 is the glue filling space, formed after the ring is mounted in the spacing ring groove, of the present invention;

FIG. 4 is a schematic diagram of the elastic die clamped by the charging electrodes and the spacing columns;

FIG. 5 is a schematic structural diagram of the ring outer shell of the present invention.

In the figures: 110, ring outer shell; 111, ring outer shell cavity; 120, ring inner shell; 130, flexible printed circuit board; 131, electronic element, 1321, No. 1 charging metal electrode; 1322, No. 2 charging metal electrode; 140, insulating element; 150, battery; 210, supporting frame; 220, No. 1 elastic die; 221, spacing ring groove; 222, glue filling space; 223, No. 2 elastic die; 211, spacing part; 2111, No. 1 spacing column; 2112, No. 2 spacing column.

### DETAILED DESCRIPTION OF EMBODIMENTS

The following clearly and completely describes the technical solutions in embodiments of the present invention in conjunction with the drawings in the embodiments of the present invention. Obviously, the described embodiments are only a part of the embodiments of the present invention, rather than all embodiments. Based on the embodiments of the present invention, all other embodiments obtained by a person skilled in the art without inventive work shall fall within the scope of protection of the present invention.

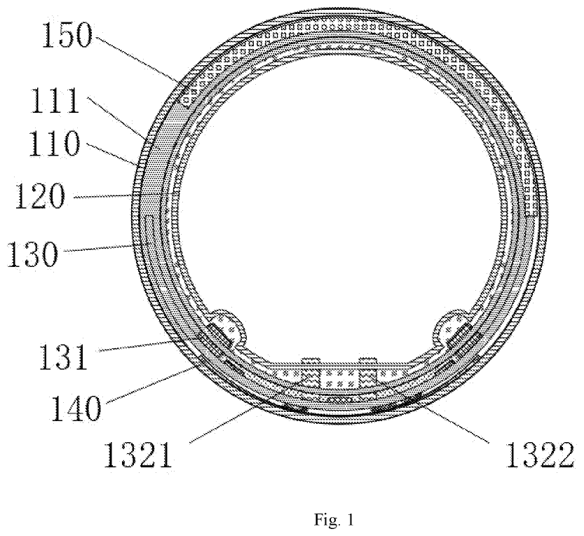

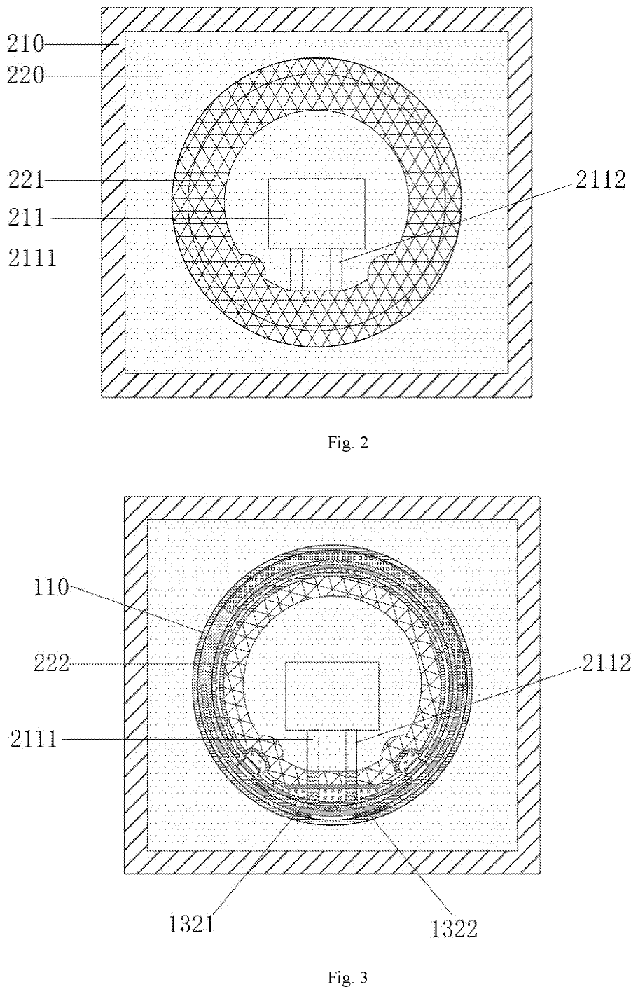

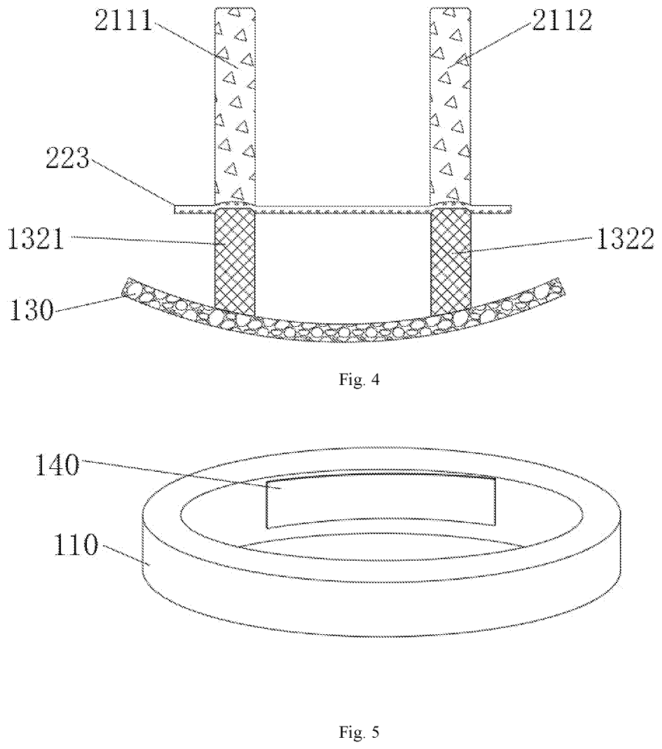

Please refer to FIG. 1-5. The present invention provides a technical solution: a smart ring containing electrodes, comprising
a ring outer shell 110 which is a ring with an inner opening;
a ring inner shell 120 which is arranged inside the ring outer shell 110, and fits closely a ring outer shell cavity 111 and an internal electronic element 131 to form a closed space. Two bulges are formed outside the ring inner shell 120 in the embodiment, and part of circuit structures penetrates the inner shell and is exposed to the environment;
the ring outer shell cavity 111 which is arranged between the ring outer shell 110 and the ring inner shell 120 and used for fixing and sealing the ring outer shell and the internal electronic element 131;
a flexible printed circuit board 130 which is mounted inside the ring outer shell cavity 111 and used for being cooperating with the operation of various electrical components;
electronic elements 131 which are arranged on the flexible printed circuit board 130 and comprise a sensing component, a processing component, a Bluetooth component, and a charging component;
a battery 150 which is arranged inside the ring outer shell cavity 111, and electrically connected with the flexible printed circuit board 130 to supply power to the flexible printed circuit board 130.

In one embodiment of the present invention, in order to ensure the durability of the ring outer shell 110, the ring outer shell can be made of rigid materials. The material can be titanium, steel, copper, gold, silver and various alloys or ceramic non-metallic materials; in order to take into account waterproofness and wearing comfort, the ring inner shell 120 is formed by permeability biocompatible flexible glue through injection molding. The flexible glue can be epoxy resin, ethylene, cyclohexene oxide or a mixture low-temperature plastic material made of various materials.

Further, the charging component includes a charging management unit, a No. 1 charging metal electrode 1321 and a No. 2 charging metal electrode 1322, wherein the No. 1 charging metal electrode 1321 and the No. 2 charging metal electrode 1322 are symmetrically arranged on the outer surface of the flexible printed circuit board 130.

In the embodiment, the smart ring adopts magnetic charging metal electrodes, and can also adopt electrocardiogram or electrodermal detection electrodes, and the No. 1 charging metal electrode 1321 and the No. 2 charging metal electrode 1322 are welded on the flexible printed circuit board 130; in order to ensure good contact during charging, the heights of the No. 1 charging metal electrode 1321 and the No. 2 charging metal electrode 1322 are required to be greater than the thicknesses of the ring outer shell cavity 111, and after glue filling is completed, the electrodes are required to be exposed outside the ring inner shell 120.

Further, an insulating element 140 is mounted on the inner wall of ring outer shell 110, and positioned inside the ring outer shell cavity 111 close to the flexible printed circuit board 130, to prevent short circuit between the electronic elements 131 on the flexible printed circuit board 130, and the ring outer shell 110.

Further, the insulating element 140 covers the area accessible to the flexible printed circuit board 130, and used for isolating the flexible printed circuit board 130 from the smart ring outer shell 110. To prevent short circuit of the electronic elements 131 on the flexible printed circuit board 130, caused by the ring outer shell 110, the insulating element 140 is mounted on the inner wall of the ring outer shell 110. In order to ensure that the mounting area covers the area accessible to the flexible printed circuit board 130, and the overall thickness of the ring is not affected, the insulating element 140 can be made of insulating glue or ultra-thin insulated rubber tape.

A glue filling jig for a smart ring containing electrodes, comprising
a supporting frame 210 which has a rectangular cavity with a top opening;
a No. 1 elastic die 220 which is arranged inside the supporting frame 210; the No. 1 elastic die 220 can be made of elastic materials, such as natural rubber, butadiene rubber and silica gel material; a spacing ring groove 221 is formed on the No. 1 elastic die 220 and used for loading an unsealed smart ring; a glue filling space 222 is formed between the unsealed smart ring and the inner wall of the spacing ring groove 221 and used for feeding glue and playing a role of plasticity in the ring inner shell 120;
a spacing ring groove 221 which is formed inside the No. 1 elastic die 220 and used for loading an unsealed smart ring;
a glue filling space 222 which is formed between the unsealed smart ring and the inner wall of the spacing ring groove 221 and used for feeding glue and shaping the ring inner shell 120;
a spacing part 211 which is arranged on the geometric center of the supporting frame 210.

Further, a No. 1 spacing column 2111 and a No. 2 spacing column 2112 are arranged on the spacing part 211, wherein a No. 2 elastic die 223 is arranged at one end of the No. 1 spacing column 2111 and one end of the No. 2 spacing column 2112; the No. 1 charging metal electrode 1321 and the No. 2 charging metal electrode 1322 on the unsealed smart ring is in close contact with the No. 2 elastic die 223 on the inner wall of the spacing ring groove 221 under the action of the No. 1 spacing column 2111 and the No. 2 spacing column 2112, to prevent glue from sealing the upper sealing surfaces of the No. 1 charging metal electrode 1321 and the No. 2 charging metal electrode 1322, and completely eradicate bad contact of charging electrodes during the charging process.

Further, the spacing part 211 and the inner wall of the supporting frame 210 form a spacing clamping groove, which performs spacing on the elastic dies and meanwhile blocks the glue flowing path in corresponding positions.

A packaging process of a smart ring containing electrodes, comprising the following steps: mounting the insulating element 140 on the inner wall of the ring outer shell, and ensuring that the mounting area covers the area accessible to the flexible printed circuit board, and the mounting method can be to paste ultra-thin insulated rubber tape or brush insulating glue;

in order to reduce remaining bubbles in the ring inner shell 120, the glue needs to be defoamed before glue filling, and the above glue filling jig needs to be dried; dispense glue in a corresponding position of the flexible printed circuit board 130 on the inner side of the ring outer shell through a dispensing process, and the flexible printed circuit board 130 and the battery 150 are fixed in the ring outer shell cavity 111;
mounting the unsealed smart ring in the glue filling jig as claimed in any one of claims 1 to 9, that is, after securing the flexible printed circuit board 130 to the ring outer shell, place it in the elastic die; a glue filling space 222 is formed among the elastic die, the ring outer shell and the flexible printed circuit board 130; after the assembly is completed, place the whole body into the supporting frame 210, fix the whole body, and apply a certain pressure to the electrode position required to be exposed through a spacing block to cut off the flowing channel between the electrode and the elastic die. Fill glue to the glue filling space 222 of the glue filling jig with a special syringe filled with glue, to ensure that the glue completely fills the inner cavity of the ring outer shell;
leaving the jig standing for hours after glue filling is completed, fixing the flexible printed circuit board 130 and exposing part of the electrodes, and performing demolding to form a glue ring inner shell 120 after the glue is completely reacted and solidified.

In the description of the present invention, it shall be understood that orientations or position relationships indicated by terms, such as “coaxial”, “bottom” “one end”, “top”, “middle” “the other end”, “above”, “one side”, “top”, “inside”, “front”, “center”, “both ends” and “geometric center”, are based on the orientations or position relationships shown in the drawings, are merely intended to describe the present invention and simplify the descriptions, but are not intended to indicate or imply that the indicated device or element shall have a specific orientation or be formed and operated in a specific orientation, and therefore cannot be understood as a limitation to the present invention.

In addition, the terms such as “first”, “second”, “third” and “fourth” are merely intended for a purpose of description, and shall not be understood as an indication or implication of relative importance or implicit indication of a quantity of indicated technical features, so that the features defined by “first”, “second”, “third” and “fourth” can expressly or impliedly contain at least one of the features.

In the present invention, unless otherwise specifically specified and defined, terms, such as “mounted”, “arranged”, “connected”, “fixed” and “spirally connected”, shall be understood in a general sense. For example, these technical terms may be a fixed connection, a detachable connection, or an integrated connection; may be a mechanical connection or an electrical connection; may a direct connection, or an indirect connection by using an intermediate medium, or may be an internal communication of two elements or an interactive relationship of two elements, unless otherwise specially defined. The person skilled in the art can understand the specific meanings of the terms in the present invention according to specific circumstances.

Although the examples of the present invention have been shown and described, the person skilled in the art can understand that various changes, modifications, substitutions and variations can be made to these examples without departing from the principle and spirit of the present invention. The scope of the present invention is defined by the appended claims and their equivalents.

What is claimed is:

1. A smart ring containing electrodes, characterized by comprising
a ring outer shell (110) which is a ring with an inner opening;
a ring inner shell (120) which is arranged inside the ring outer shell (110);
a ring outer shell cavity (111) which is the cavity defined by the ring outer shell (110) and the ring inner shell (120);
a flexible printed circuit board (130) which is mounted inside the ring outer shell cavity (111);
electronic elements (131) which are arranged on the flexible printed circuit board (130) and comprise a sensing component, a processing component, a Bluetooth component, and a charging component;
a charging component which is fixedly connected with the outer side of the flexible printed circuit board (130); the height of the charging component is greater than the thickness of the ring outer shell cavity (111); after glue filling is completed, electrodes of the charging component are exposed outside the ring inner shell (120);
a battery (150) which is arranged inside the ring outer shell cavity (111), and electrically connected with the flexible printed circuit board (130) to supply power to the flexible printed circuit board (130).

2. A smart ring containing electrodes according to claim 1, characterized in that the charging component comprises a charging management unit, a No. 1 charging metal electrode (1321) and a No. 2 charging metal electrode (1322) which are symmetrically arranged on the outer surface of the flexible printed circuit board (130).

3. A smart ring containing electrodes according to claim 1, characterized in that an insulating element (140) is mounted on the inner wall of ring outer shell (110), and positioned closely to the flexible printed circuit board.

4. A smart ring containing electrodes according to claim 1, characterized in that a mounting area of the insulating element (140) covers the area accessible to the flexible printed circuit board (130), blocking the electrical connection between the electronic components (131) and the ring outer shell (110), and the insulating element (140) is made of insulating glue or ultra-thin insulated rubber tape.

5. A smart ring containing electrodes according to claim 1, characterized in that the ring outer shell (110) is made of one of titanium, steel, copper, gold, silver, alloys or ceramic non-alloys, and the ring inner shell (120) is made of flexible glue which can be composed of oxygen resin, polyethylene, cyclohexene oxide or a type photosensitive adhesive.

6. A glue filling jig for a smart ring containing electrodes according to any one of claims 1 to 5, characterized by comprising a supporting frame (210) which has a rectangular cavity with a top opening;
a No. 1 elastic die (220) which is arranged inside the supporting frame (210);
a spacing ring groove (221) which is formed inside the No. 1 elastic die (220) and used for loading the smart ring that has not been sealed with glue;
a glue filling space (222) which is formed between the unsealed smart ring and the inner wall of the spacing ring groove (221) and used for feeding glue and shaping the ring inner shell;
a spacing part (211) which is arranged on the geometric center of the supporting frame (210).

7. A packaging process of a smart ring containing electrodes, characterized by comprising the following steps:
mounting the insulating element (140) in a corresponding area of the inner wall of the ring outer shell;
dispensing glue in a corresponding position of the flexible printed circuit board (130) on the inner wall of the ring outer shell through a dispensing process, and fixing the flexible printed circuit board (130) and the battery (150) in the ring outer shell cavity;
mounting the unsealed smart ring in the glue filling jig, and filling glue to the glue filling space (222) of the glue filling jig with a special syringe filled with glue, to ensure that the glue completely fills the inner cavity of the ring outer shell;
leaving the jig standing for hours after glue filling is completed, securing the position of the flexible printed circuit board and exposing part of the electrodes, and performing demolding to form a glue ring inner shell after the glue is completely reacted and solidified.
